(19) United States
(12) Patent Application Publication
(10) Pub. No.: US 2013/0216112 A1
(43) Pub. Date: Aug. 22, 2013

(54) STRUCTURED, IMAGE-ASSISTED FINDING GENERATION

(71) Applicant: Joachim Graessner, Boenningstedt (DE)

(72) Inventor: Joachim Graessner, Boenningstedt (DE)

(21) Appl. No.: 13/768,185

(22) Filed: Feb. 15, 2013

(30) Foreign Application Priority Data
Feb. 17, 2012 (DE) 102012202447.6

(51) Int. Cl. A61B 5/00

(52) U.S. Cl. CPC A61B 5/00
USPC 382/128

(57) ABSTRACT

In a method for visual referencing of image information within the scope of a workflow control in an image-assisted medical finding, as well as a data structure, a workflow control system with a control module, and a non-transitory storage medium encode with programming instructions, in addition to an image to be assessed, at least one reference image is superimposed at a monitor in which the most frequent pathological variations of the body region shown in the image are presented in order to enable a uniform referencing.

### BACKGROUND OF THE INVENTION

1. Field of the Invention

The invention lies in the field of medical engineering and informatics, and in particular concerns the image-assisted assessment of magnetic resonance tomography (MRT) exposures or exposures from other modalities.

2. Description of the Prior Art

Within the scope of image-assisted medical finding, a number of image data sets must normally be viewed, analyzed and assessed to generate a medical report. The image data sets can originate from the same patient but from different acquisition points in time, or they can have been acquired with different image acquisition apparatuses (MRT, CT, etc.). This hinders the ability to compare the data sets to be assessed.

In today's modern systems, the finding system is computer-based and imports data from one or more acquisition systems via an interface. A radiologist who can access the acquired image data sets via a network works at the finding workstation, which is normally physically and spatially separate from the acquisition system. The radiologist can access the acquired image data with the use of a picture archiving and communications system (abbreviated PACS in the following) from his or her computer-based workstation (normally arranged in a radiology department or in a radiology practice of a physician in private practice).

The user conventionally implements the finding at such a computer workstation (for example at a viewing workstation of a clinic department, for example radiology). He or she must analyze the anatomical or other structures (knee, in particular meniscus, for example) displayed with the image data and implement a comparison with a normative and/or pathological state of the respective structure.

A significant disadvantage of previous systems is that there is no standardized procedure. The results thus tend to be subjective and poorly comparable, which reduces the quality of the generated report. Furthermore, the viewer not provided with a visual structure on which the viewer can orient himself or herself and/or receives only minimal assistance in that regard.

### SUMMARY OF THE INVENTION

An object of the present invention is to improve and standardize the workflow control in an image-assisted, medical finding. Furthermore, a method for optical referencing that can be used within the scope of the report generation should be automated and improved. Conventional computer-based finding systems are to be improved and in particular to be expanded by a control module. Furthermore, a normalizable control of the workflow of a process within the scope of the workflow of the finding should be possible.

This object is achieved in accordance with the invention by a computer-based method for visual referencing, a workflow control system with a control module, such a control module itself, and a non-transitory, computer-readable data storage medium encoded with programming instructions.

In the following, the achievement of the object is described with regard to the method. Features, advantages and/or alternative embodiments are likewise applicable to the other aspects of the invention. In other words: the workflow control system, the data structure and/or the programming instructions can also be developed with the features that are described in connection with the method. The corresponding functional features of the method are formed by corresponding objective computer-implemented modules, in particular microprocessor modules of the system. The workflow control system can also be integrated as an embedded system into the acquisition system and/or into a workstation (the finding system, for example).

According to one aspect, the invention concerns a method for optical referencing of image data that must be processed within the scope of an image-assisted medical finding, and a workflow control in this regard, that includes the following steps:
display an image with one or more anatomical region(s) (knee joint, for example) at a display device (at a monitor or finding console, for example)
automatic detection of an identifier for the displayed anatomical region
superimpose at least one reference image at the detected identifier that serves as a reference (or, respectively, as a comparison metric) with regard to the likewise displayed anatomical region.

A structure that can be used for workflow control of a finding process is provided with the additionally superimposed reference image. The finding process thus can be controlled uniformly using a predefined workflow structure, and thus can also be made objective for different users and/or systems (for example even internationally or across clinics).

A series of advantages that prove to be relevant in practice is achieved with the achievement according to the invention.

As mentioned above, overall it proves to be very advantageous and improves quality that the individual analysis processes can be made comparable within the scope of the finding. A uniform workflow structure is defined that must (can) then be applied to all processes.

An additional advantage is that an inexperienced user can also access this same database to classify the image data (and thus for referencing) as an experienced assessor with extensive experience. Furthermore, the results of the finding process (for example in text form as a report) can also be passed directly into other computer-based systems (for example, in the syngo.via system from Siemens AG the results are immediately sent to what is known as the Findings Navigator and imported there).

The terms used within the scope of this application are explained in detail in the following.

The method is typically installed entirely or partially at a finding system. The finding system is a computer workstation of the radiologist. The radiologist typically operates in a radiology department that can also be located far from the respective imaging apparatus. The assessment of the image data acquired by means of an acquisition system takes place at a separate, specific workstation of the radiologist after the images have been transferred to the respective computer via an interface. A client of a radiological finding software is typically installed at the finding computer. According to a preferred embodiment, this is a client of the syngo.via client/server system. This system is designed for viewing, analysis or evaluation and storage of the medical images.

The term “referencing” should be understood within the scope of a comparison. The referencing is in particular based on image data. For a finding or to create a medical report, the current case data (for example the current image of the examined knee of the patient) must be compared with comparison data (healthy knee and/or typical, pathologically altered knee) for coincidence and/or deviation. A metric is applied for this purpose. According to the invention, this process is standardized insofar as that it can be ensured that a uniform comparison scale and/or a uniform database for the reference images can always be applied.

The finding is image-based. Image information for assessing the current case is thus typically presented on a monitor or other display device of the finding system. The invention thus in principle can be applied to all different image acquisition apparatuses such as MRT (magnetic resonance tomography), CT (computed tomography), conventional x-ray systems with x-ray images, US (ultrasound), PET (positron emission tomography) or other (among these also functional) imaging methods. Moreover, the image data can also comprise additional metadata that are likewise presented partially or in a selected form (for example metadata of the image data about the patient, age, gender, acquisition point in time etc.)

The term “anatomical region” is used herein comprehensively and refers to body regions or body structures of a patient that have been examined or, respectively, measured by means of an imaging method. The anatomical region is represented in the image. It can be a joint, an organ or their regions or segments, for example multiple individual or contiguous regions of a pathologically altered liver. The image can be a 2-dimensional or 3-dimensional representation. It is likewise possible to display the image as a 4-dimensional data set (for example as a video or film). The reference images are typically superimposed with the same dimensions in order to ensure an optimally good coincidence and comparison capability. However, it is also possible that the format differs between image and reference image, such that only 2-D reference images are superimposed for a 4D image. Moreover, the anatomical region displayed in the image can also include physiological values. The images are advantageously processed and displayed in a special format, namely in the DICOM format (DICOM: Digital Imaging and Communications in Medicine). In the DICOM format, the image data are divided up into two categories: actual pixel data and metadata. Among other things, the metadata comprise an orientation of the image (for example transversal, sagittal, coronal/frontal etc., possibly with additional spatial designations) as an image label and/or a DICOM attribute “body part examined”. The respective organ or the respective anatomical structure (for example patella, right) can then be automatically derived from these metadata. However, the invention is not limited to such a protocol and, for example, can alternatively comprise other network protocols (for example Internet-based protocols such as http/ip or the like). The interface is designed to exchange image data, control commands and/or identifier data. The type of data transfer is not limited in principle. However, it is normally provided that the image data, control commands and/or identifier data are transferred as separate messages via the interface. Alternatively, they can also be bundled and transferred in combination in a common packet (as a message packet).

The identifier characterizes the content of the displayed image, and in particular the anatomical structure (for example in the orthopedic application case: knee joint with meniscus). The identifier is a digital data set that advantageously uniquely identifies the structure at the core of the examination or image acquisition. Reference images (thus for example comparison images of healthy and/or pathologically altered knee joints) can then be found and provided in a data structure via the identifier.

The reference image can be provided as a single comparison image or as a set of images. This has as its content the same anatomical structure as the displayed image (the image to be assessed). A significant aspect of the invention is apparent in that the superimposition of the reference image is executed automatically (thus without a user interaction). In particular, the recognition (the detection) of the identifier also takes place automatically and/or on the basis of a DICOM attribute associated with the image (for example “body part examined”). The reference image or the group of reference images is advantageously presented simultaneously or in parallel with the image at the display device. The user therefore can individually compare the displayed image (the image to be assessed) with the reference image(s) in a screen presentation.

The reference image is superimposed (overlaid) at the display device; the overlay time can be preset. The overlay can be triggered at a predefinable user signal, for example when the mouse or another UI device is moved over the displayed image (mouse hover, mouseover). Alternatively, a presetting can be made so that the reference image remains shown for a predetermined time period, advantageously in a separate window. The time period is advantageously preset so that it coincides with the display time for the image so that the reference images are displayed at most as long as the image itself, and not any longer. This has the advantages that the method can be adapted dynamically to the requirements of the user and situation, and that it can be set so that the user is not unnecessarily confronted with auxiliary information, but only for a sufficient length of time.

In an alternative embodiment, the reference image is superimposed on the image in a transparent but visible presentation so that differences between image and reference image are visible immediately and at a glance. However, it must be ensured that the (original, to be assessed) image remains completely visible. This has the advantage that the user can recognize the differences even more simply and quickly. Here an automatic size adaptation and orientation adaptation to the respective case advantageously take place. The reference image is thus subject to an automatic transformation process so that it can be presented in approximately the same orientation and/or size as the image.

Depending on the selected embodiment of the invention, the reference image can have different structures as content. The reference image can be an image showing at least one pathological state of the anatomical region. In the preferred embodiment, the most frequent forms of injury to the structure (in the knee example above: medial tear of the meniscus, partial tear, longitudinal tear, dislocation etc.) are selected and presented as a reference image according to a preconfigurable statistical criterion. This has the advantage that the user is not confronted with an unnecessarily large number of comparison presentations. The reference image can also be an image showing a healthy version of the anatomical structure (healthy knee joint). The certainty of the system can therefore be increased in that the user receives the opportunity to compare the current body state with healthy/normal states in order to also eliminate smaller lesions or injuries with a greater certainty. It is likewise possible for the reference image to include textual data that identify the typical injury forms of the respective displayed anatomical regions. This can assist the assessor in simply and quickly making a description of the lesion (for example, given meniscus injury: dislocation, partial tear, initial tear etc.). These text data are likewise superimposed at the monitor, and the user can select individual entries via user interaction (for example a mouse click) and integrate them into his report.

According to a further development of the invention, a data structure in which at least one reference image is associated with a respective image via an identifier is accessed to search for the at least one reference image. The data structure can be provided at the finding system or be accessible via a network. This has the significant advantage that the association between image and reference image can be adapted dynamically. Given new finding results, these can even be mapped in the data structure in order to thus already be provided immediately to all following examinations and findings. The modularity of the system can likewise be increased via the separate provision of the data structure. The association can thus also be changed at any time (for example from a central location).

The chronological sequence of the method steps—display the image, detect the identifier and superimpose the reference image—does not necessarily need to be formed sequentially [sic] as the naming of the steps possibly suggests. The steps can also overlap in time or even be executed simultaneously.

Moreover, it is possible that individual segments of the method described in the preceding can be designed as individual salable units, and remaining segments of the method can be designed as other salable units. The method according to the invention can therefore be executed as a distributed system at different computer-based instances (for example client/server instances). For example, it is thus possible that the control module for its part comprises different sub-modules that are implemented in part at a central system, in part at the finding system and/or in part at other computer-based instances.

The invention encompasses a data structure to store a mapping table. The data structure can be formed directly in a memory of the finding system or be accessible as a separate instance and via a network connection. The data structure includes the mapping table with an association between an anatomical region (that is addressed and accessible via the identifier) and at least one reference image.

Furthermore, the invention concerns a workflow control system for image-assisted medical assessment, which includes:
a display device that is designed to display an anatomical region of an image and to superimpose at least one reference image,
a data structure in which a mapping table is stored with an association between a respective image and at least one reference image, wherein the image and/or reference image can be addressed via an identifier,
a control module that can be implemented in a calculation unit and serves to enable a preconfigurable comparison standard via the overlaying of reference images associated with the respective image.

The data structure and the control module can be implemented at the same computer-based instance. The control module is a computer-based module. It can be designed as a software module or as a hardware module (as a module of a microprocessor). The control module serves to expand the finding system. The control module is advantageously integrated directly into the finding system and can also be provided as an embedded system at the finding system. In an alternative embodiment, the control module is not directly integrated into the finding system but rather is provided as a separate instance. The control module can then be executed at a separate computer-based instance that, for example, can be connected to the finding system via an interface.

The present invention also encompasses a non-transitory, computer-readable data storage medium encoded with programing instructions but, when executed by a computer, cause any or all embodiments of the method described above to be implemented.

The instructions are loaded into and stored in a memory of a computer and include computer-readable commands that are designed to cause the method described in the preceding to be implemented when the commands are executed by the computer. The programming instructions can also be stored at a storage medium or can be downloaded from a server via an appropriate network.

### BRIEF DESCRIPTION OF THE DRAWINGS

FIG. 1 is a schematic presentation of a medical finding system that is expanded with a control module according to a preferred embodiment of the invention.

FIG. 2 is a workflow diagram of the method according to the invention according to a preferred embodiment of the invention.

### DESCRIPTION OF THE PREFERRED EMBODIMENTS

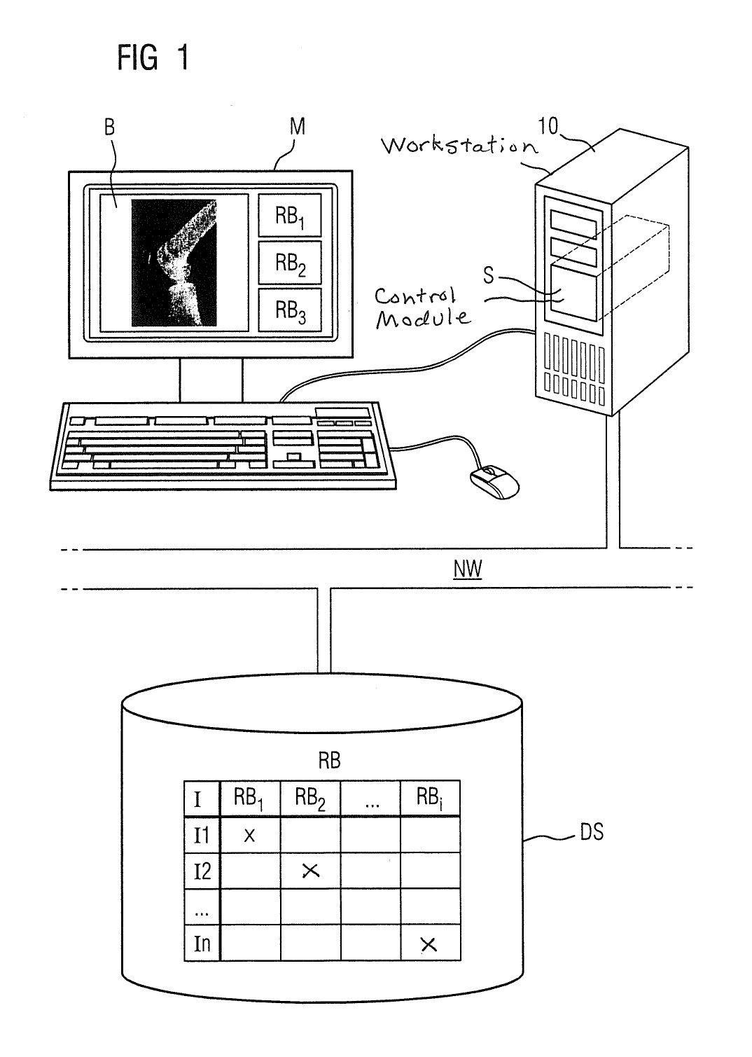

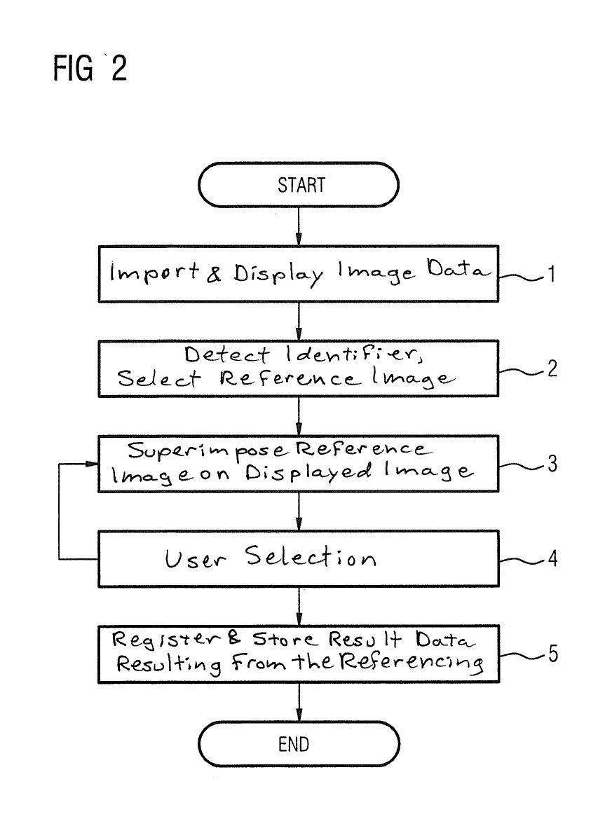

In the following the invention according to preferred embodiments are explained in detail with reference to FIGS. 1 and 2.

As can be seen from FIG. 1, a finding system has a monitor M that is connected with a workstation 10 via corresponding interfaces. Additional devices (such as mouse and keyboard) are provided as input and output interface. Images B of a patient that are to be assessed (for example the image of a knee given a knee injury as this should be indicated in FIG. 1) are displayed on the monitor M.

According to the invention, the workstation 10 is expanded with a control module S. The workstation 10 is engaged in data exchange with a data structure DS (in which a mapping table is stored) via a network NW. The mapping table comprises entries that are uniquely addressable via an identifier I. The entries in turn comprise images and reference images. All associated reference images RB can be found via the association with an image B.

Anatomical structures such as organs (for example heart, liver, spleen, lung etc.) or organ parts are represented in the images B and/or reference images RB. Individual (for example broken or otherwise damaged) body structures (for example knee joint, ulna, radius, bones of the leg etc.) could likewise be represented as an anatomical region in the image B in surgical and/or orthopedic application cases. The image B is acquired with an imaging acquisition apparatus (CT, MRT, ultrasound etc., for example) or imported via a data interface. In addition to the pure pixel data, the image data thereby also comprise metadata in which the examined body region of the patient is defined in detail; for example, the metadata comprise data regarding gender, age and additional data of the patient, acquisition point in time, type of acquisition (for example contrast agent-assisted mammography), identification of the examined organ/body structure. For example, given x-ray exposures an identification set for the patient and the type of acquisition (for example meniscus, right, sagittal, date) is always located together at the image B. For example, in the DICOM format an attribute (“body part examined”) that identifies the examined body region is carried as well. This attribute can then be used as an identifier. Alternatively or in addition, other identification data sets (image orientation, inherited identifiers from the study or series associated with the image) can be used as an identifier. The body region or the body structure depicted in the image B can be uniquely identified via the identifier.

At least one instance of reference images RB is now stored in the data structure DS with regard to a respective identifier I. A set of reference images RB is stored with regard to an identifier I (in FIG. 1, RBi is associated with the identifier I1, RB₁ is associated with the identifier I2, . . . , RBi is associated with the identifier I1.

The reference images RB are images that pertain to the same anatomical region as the image B that, however, have a different status (healthy, degeneratively altered in multiple stages, typical pathological variation etc.). Other versions of the same body region can also be used as a reference image RB (for example reference image of the same organ/region at a different stage of life given different basic illnesses etc.). The reference images RB can be adapted to new knowledge at any time. They should serve as a comparison scale for the image B to be assessed. For example, the physician can therefore more easily determine whether a bone deformation determined and shown in image B is a typical change given arthrosis (as is then apparent from the superimposed reference images RB) or a different incurred deformation.

An important aspect of the present invention is apparent in assisting the user in his activity and providing to him a control structure or workflow structure as a standard on which he can orient himself. In particular, he can handle his finding task by resorting to a centrally stored database that serves as a metric for his evaluation. It can therefore be ensured that two different physicians apply the same assessment criteria in different clinical units (possibly even across international borders) in that the same basis for comparison is considered with the same comparison images.

In the following, a workflow according to a preferred embodiment of the invention is described in detail with reference to FIG. 2.

After the start of the system, in Step 1 the image data of the acquisition system are imported and presented at the monitor M of the finding computer 10. For this the data are imported via a provided interface between acquisition system and finding system. The message exchange can thereby be selectively initiated by the acquisition system or by the finding system.

The detection of the identifier I from the anatomical region shown in image B takes place in Step 2. This is advantageously automatic and can be executed by reading out a DICOM attribute. Alternatively, here the user can also make a selection from a list that is displayed to him (semi-automatic registration) or a manual input (manual registration).

After the registration of the identifier I, an access to the data structure DS is executed in order to find all reference images RB that are associated with the identifier I.

All or selected assessed reference images RB are then superimposed at the monitor M in Step 3.

The method can end here. In an embodiment of the invention that is shown in FIG. 2, in Step 4 the user can select from the set of reference images RB a few reference images RB as relevant so that only the relevant reference images RB are then superimposed on the monitor. This embodiment has the advantage that the user is not diverted or disrupted by unnecessary, confusing information.

Result data of the referencing can be registered and (optionally) stored in Step 5. The result data are related to the reference images RB that are selected or determined by the user as coinciding. This has the advantage that the basis for the assessment exists for the same assessor, or also a different assessor—possibly also at a later point in time. The method then ends.

The invention can be briefly summarized as follows.

The invention implements an automatic superposition of reference images RB (identified as relevant) with regard to an image B to be assessed. A uniform metric can therefore be considered for comparison of the structures displayed in image B, metric is also uniform for different users and across clinic boundaries. The finding can thus be standardized. Furthermore, according to the invention an adaptation of the finding structure can be realized easily and simply (for instance due to technical improvements in the imaging methods).

Multiple advantages can be achieved by the solution according to the invention. It is possible to provide a control structure with a uniform assessment scale within the scope of the finding. Moreover, all relevant reference images RB can be taken into account directly and automatically, even when the reference images have been registered at other locations (other clinic or other finding system) in order to therefore improve the quality of the finding. Furthermore, the finding can also be standardized beyond clinical boundaries and for different users. The reference images RB can be superimposed automatically and directly without it being necessary to call in a medical consult with authorization measures and/or needing to activate other communication channels. This has the advantage that the finding physician can directly access remote reference images RB from his workstation without needing to leave his workstation or calling up other applications. Overall, this leads to a higher finding quality, to a more efficient finding (time savings), and increases the operating comfort.

In conclusion, it is noted that the preceding description of the invention with the exemplary embodiments is to be understood as not limiting with regard to a specific physical realization of the invention. For those skilled in the art it is clear that the invention is fundamentally not limited to MR measurements or x-ray acquisitions, but rather can be used for other imaging systems. Moreover, it is also not absolutely necessary to access DICOM-based communication technology. For example, proprietary protocols can also be used here for process communication. Moreover, the invention can be implemented partially or completely in software and/or in hardware. Moreover, the method or the control system according to the invention can also be realized distributed across multiple physical products, comprising computer program products. It is thus possible to implement a portion of the control at the finding system and a remaining portion of the control at a central instance.

Although further modifications and changes may be suggested by those skilled in the art, it is the intention of the inventor to embody within the patent warranted hereon all changes and modifications as reasonably and properly come within the scope of his contribution to the art.

I claim as my invention:

1. A method for visually referencing image information at a display of a computerized workstation, comprising:
in a processor of a computerized workstation, executing a user-interactive workflow configured to provide medical image assistance in making a medical finding;
in said workflow, causing, from said processor, display of a medical image, which contains at least one anatomical region, at a display device in communication with said processor;
in said processor, automatically detecting an identifier that identifies said at least one anatomical region in the displayed image;
in said processor, accessing a databank in which a plurality of reference images are stored and, using said identifier, selecting at least one of said reference images that provides a visual reference to a viewer at said display device for said at least one anatomical region in the image displayed at said display device; and
from said processor, automatically also causing said at least one of said reference images to be displayed with said at least one anatomical region of said image at said display device.

2. A method as claimed in claim 1 comprising, in said processor, detecting said identifier by automatic detection of content of said at least one anatomical region in said image displayed at said display device.

3. A method as claimed in claim 1 comprising providing information in a DICOM header associated with said image displayed at said display device, and automatically detecting said identifier in said processor based on said information in said DICOM header.

4. A method as claimed in claim 1 comprising, in said processor, detecting said identifier from an orientation of said at least one anatomical region in said image at said display device.

5. A method as claimed in claim 1 comprising presenting said at least one of said reference images simultaneously or in parallel with said image at said display device.

6. A method as claimed in claim 1 comprising maintaining display of said image at said display device with no reference image superimposed thereon, and superimposing said at least one of said reference images on said image at said display device in a separate window at said display device.

7. A method as claimed in claim 1 comprising selecting said reference image from the group consisting of at least one pathological version of said at least one anatomical region in said image at said display device, and at least one healthy version of said at least one anatomical region in said image at said display device.

8. A method as claimed in claim 1 comprising storing said reference images in said databank respectively with an associated data structure that causes respective reference images to be selected based on said identifier.

9. A workflow control system for implementing an image-assisted medical finding, comprising:
a processor configured to execute a user-interactive workflow configured to provide medical image assistance in making a medical finding;
a display device in communication with said processor;
said processor in said workflow, being configured to cause display of a medical image, which contains at least one anatomical region, at said display device;
said processor being configured to automatically detect an identifier that identifies said at least one anatomical region in the displayed image;
said processor being configured to access a databank in which a plurality of reference images are stored and, using said identifier, to select at least one of said reference images that provides a visual reference to a viewer at said display device for said at least one anatomical region in the image displayed at said display device; and
said processor being configured to automatically cause display of said at least one of said reference images with said at least one anatomical region of said image at said display device.

10. A non-transitory, computer-readable data storage medium encoded with programming instructions, said data storage medium being loaded into a computerized processor, in communication with a display device, and having access to a databank, said programming instructions causing said processor to:
execute a user-interactive workflow configured to provide medical image assistance in making a medical finding;
in said workflow display a medical image, which contains at least one anatomical region, at said display device;
automatically detect an identifier that identifies said at least one anatomical region in the displayed image;
access said databank, in which a plurality of reference images are stored and, using said identifier, select at least one of said reference images that provides a visual reference to a viewer at said display device for said at least one anatomical region in the image displayed at said display device; and
automatically cause said at least one of said reference images to be displayed with said at least one anatomical region of said image at said display device.

11. A storage medium as claimed in claim 10 wherein said programming instructions cause said reference images to be stored in said databank each with an associated data structure that allows a respective reference image to be selected based on said identifier.
